(19) United States
(12) Patent Application Publication
(10) Pub. No.: US 2002/0133128 A1
(43) Pub. Date: Sep. 19, 2002

(54) Method and apparatus for dilating an orifice in biological tissue

(76) Inventor: Andrew S. Heller, Vineland, NJ (US)

Correspondence Address: Gregory S. Bernabeo, Esq., Synnestvedt & Lechner LLP, 2600 Aramark Tower, 1101 Market Street, Philadelphia, PA, 19107-2950, US

(21) Appl. No.: 09/810,793

(22) Filed: Mar. 16, 2001

(51) Int. Cl. A61M 29/00

(52) U.S. Cl. 604/270

(57) ABSTRACT

A dilation kit for dilating an orifice in living tissue. The kit includes at least a guidewire, a first dilator defining an internal throughbore for receiving the guidewire, and a second dilator defining an internal throughbore for receiving the guidewire. The second dilator defines an outer surface having a periphery greater than the periphery of the first dilator. In one embodiment, the straightening element is part of a guidewire advancement device. The kit may also include a hemostat and/or a PEG tube. A method for using the dilation kit to dilate an orifice in living tissue is also provided.

### FIELD OF THE INVENTION

The present invention is directed generally to a method and apparatus for dilating an orifice in biological tissue. In particular, the present invention is directed to a method and apparatus for using a guidewire technique to dilate and orifice in biological tissue to facilitate replacement of a percutaneous, endoscopic gastrostomy tube.

### BACKGROUND OF THE INVENTION

Patients with digestive tract problems are often fitted with percutaneous, endoscopic gastrostomy (“PEG”) tubes. Such PEG tubes are inserted by a surgical procedure wherein an orifice is formed in the skin fascia and stomach wall and whereby the PEG tube is mounted through the orifice to allow nutrition to be provided through the PEG tube and directly into the patient's stomach and intestines. Examples of individuals who would require such a procedure include burn patients, whose daily caloric needs are very high, critically ill, weak or comatose patients who may be unable to chew their food, and patients suffering from a diseased or traumatized esophagus who may be unable to swallow food. Exemplary gastrostomy tubes are disclosed in U.S. Pat. No. 4,668,225 to Russo et al., U.S. Pat. No. 4,758,219 to Sacks et al., U.S. Pat. No. 5,080,650 to Hirsch, et al., and U.S. Pat. No. 5,807,314 to Ross et al., the disclosures of which hereby are incorporated herein by reference.

PEG tubes often become dislocated from the patient's body, e.g. due to restlessness while sleeping, or because the patient has intentionally removed the tube despite the patient's need for it. If dislocation of the tube is discovered promptly, a simple percutaneous insertion is possible, whereby the tube is simply reinserted into the existing orifice in the patient's abdomen. This procedure may be performed by moderately skilled personnel, such as nursing home staff, without the need for a medical doctor or surgeon, provided that the PEG tube has been carried by the patient for a sufficiently long period of time to have established a relatively durable passageway through the patient's bodily tissue, e.g. at least two months.

Unfortunately, dislocation of the PEG tube is rarely discovered promptly. Approximately two to three hours after dislocation, the orifice in the patient's abdomen begins to close, making a simple percutaneous insertion impossible. Admission of the patient to a hospital is then required. A hospital stay is distressing to the patient and requires administration of intravenous fluids and other preparations by skilled personnel. The PEG tube is then reinserted by a surgeon or gastroenterologist in due course. Involvement of a surgeon or gastroenterologist causes considerable costs to the patient and/or the patient's insurer. If dislocation of the tube occurs on a Friday night, weekend or holiday, the patient's hospital stay may extend over several days, causing considerable additional costs and distress to the patient.

In the field of vascular medicine, present methods and apparatuses exist for inserting a foreign object into a vein or body cavity in order to facilitate catheter placement. For example, the Seldinger technique, developed by Sven I. Seldinger, involves introducing a spring wire guide or “guidewire” into the vein or body cavity. Basically, the Seldinger technique involves puncturing a vein with a hollow needle and advancing the guidewire through the needle, into the vein, at which point the needle is removed. Guidewires are generally comprised of a coiled spring guide with a distal tip and one or more wires running longitudinally within the spring. Exemplary guidewire constructions are disclosed in U.S. Pat. No. 4,003,369 to Heilman et al. and U.S. Pat. No. 4,676,249 to Arenas et al., the disclosures of which are incorporated herein by reference.

Advancements in guidewire design have led to prevalent use of a J-shaped guidewire. A “J” portion of the guidewire is straightened by a straightening element. In its basic form, the straightening element is a tube with an internal bore dimensioned to receive the guidewire (see FIG. 1). As the guidewire is advanced, the “J” portion of the guidewire passes out of the straightening element and attempts to return to its J-shape. This causes the guidewire to be wedged against the walls of the vein and to be gently fixed in place.

Developments in the art have combined guidewires and straighteners and/or syringes into guidewire advancement devices. Various guidewire advancement devices are well known. Exemplary devices are disclosed in U.S. Pat. No. 4,274,408 to Nimrod, U.S. Pat. No. 4,813,938 to Rawlerson, U.S. Pat. No. 4,713,059 to Bickelhaupt et al., U.S. Pat. No. 5,484,419 to Fleck (see FIG. 2), and U.S. Pat. No. 6,011,988 to Lynch et al., the disclosures of which are incorporated herein by reference. Such guidewire advancement devices are typically used by highly skilled physicians such as international radiologists, invasive cardiologists, or surgeons. Lower-level patient care givers are prohibited from using such devices.

In accordance with the Seldinger technique, after the guidewire is in place, a catheter is threaded over the guidewire and into the vein. Catheters are generally hollow, flexible tubes used to convey liquids or other instruments to a desired location in the body. The catheters are generally very thin or of a small diameter to facilitate easy movement within the vein. The guidewire is then withdrawn, making the catheter ready for further positioning and/or use.

It has been noted that where the patient has had a PEG tube for approximately eight or more weeks, there is usually an established track through the patient's abdomen into the patient's stomach which would permit percutaneous reinsertion without any substantial risk of entering the free peritoneal space. While existing guidewire advancement devices and techniques could be used to position a guidewire within the track, they are of no assistance in replacing a PEG tube, due in part to the relatively large size of a PEG tube and the relatively small size of catheters, straightening elements and/or guidewire advancement devices.

Applicant has identified a need for a method and apparatus for dilating an orifice in biological tissue. More specifically, there is a need permitting non-surgical reinsertion of a PEG tube which may be performed by moderately skilled personnel, e.g. an Emergency Room physician, without the need for surgery or a hospital stay, even after the orifice in the patient's abdomen begins to close. The present invention fulfills these needs, among others.

### SUMMARY OF THE INVENTION

The present invention provides an apparatus, i.e. a dilation kit, for dilating an orifice in living tissue. The dilation kit includes at least a guidewire and first and second dilators. Preferably, several dilators are included. Each dilator defines an internal throughbore for receiving the guidewire and further defines an outer surface having a periphery. The peripheries of the first and second dilators have different sizes.

Optionally, the kit may include an initial dilator for stiffening and/or straightening the guidewire and/or a guidewire advancement device for inserting the guidewire into an orifice. The kit may also include a hemostat for stretching of the orifice, e.g. after dilation with a dilator, and/or a PEG tube.

A method for dilating an orifice in biological tissue using the inventive dilation kit is also provided. The method includes the steps of positioning a guidewire in the orifice, dilating the orifice by advancing a first dilator along the guidewire and into the orifice, further dilating the orifice by advancing a second dilator along the guidewire, and removing the guidewire from the orifice. Optional steps include stretching of the orifice with a hemostat and/or advancement of a PEG tube along the guidewire and into the orifice.

### DESCRIPTION OF THE DRAWINGS

FIG. 1 is an elevational view of an exemplary guidewire advancement device of the prior art;

FIG. 2 is an elevational view of the guidewire advancement of FIG. 2, showing the guidewire partially advanced;

FIGS. 3a-e are elevational views of dilators in accordance with the present invention; and

FIG. 4 is a top view of an exemplary dilation kit in accordance with one embodiment of the present invention.

### DETAILED DESCRIPTION

The present invention provides a dilation kit for dilating an orifice in living tissue. Such a kit is useful, for example, for reinserting a PEG tube into an orifice into a patient's abdomen, even after expiration of the period permitting simple percutaneous reinsertion and after partial closure of the orifice. Although the kit has many other uses, it is discussed below in the context of reinsertion of a PEG tube for illustrative purposes. The present invention provides a method an apparatus for which can be performed by moderately skilled medical personnel, e.g. by an Emergency Room physician, without the need for surgery and/or a hospital stay, thereby reducing costs to the patient and/or the patient's insurer and reducing distress to the patient.

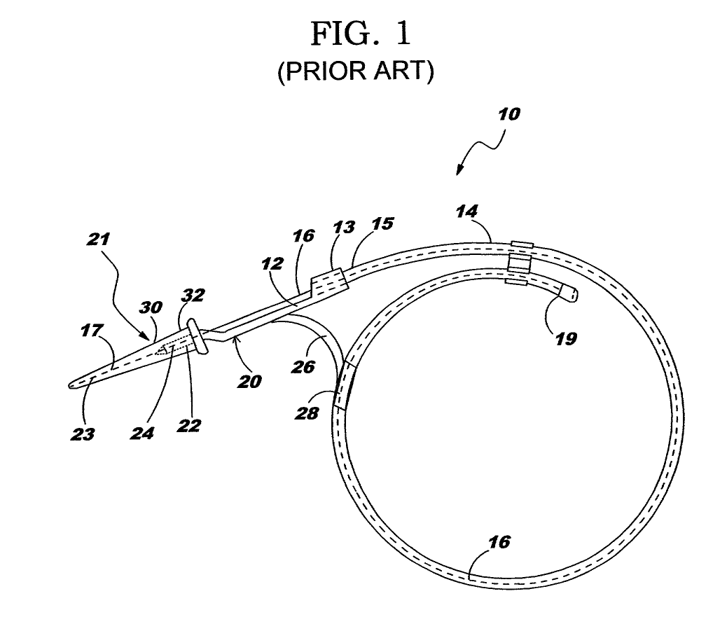

A dilation kit in accordance with the present invention includes a guidewire. Many guidewires are known in the prior art. Any suitable guidewire may be included. Referring now to FIG. 1, a “J-shaped” guidewire 16, as is known in the art, is included. The guidewire is typically made of winding of a plurality of individual metal filaments. For a dilation kit suitable for a PEG tube reinsertion, the guidewire is preferably approximately 10-25 cm in length and most preferably 20 cm in length.

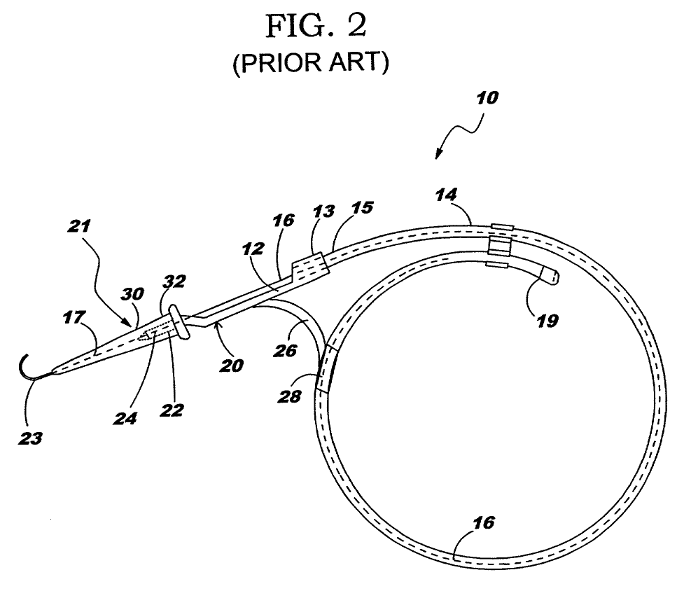

In the preferred embodiment, the kit includes a guidewire advancement device. Any suitable guidewire advancement device may be used. An exemplary guidewire advancement device 10 is shown in FIG. 1. The device 10 is preferably made of molded plastic and includes a longitudinal base 12. The longitudinal base member 12 includes an attached elevated rear end 13 which fastens to and attaches a flexible tube 14. The flexible tube 14 houses a coiled guidewire 16 (represented by the broken line of FIG. 2) and has a proximal end 15 attached to the rear end 13 of the longitudinal base 12.

A central open section 20 of the longitudinal base 12 is open and attaches to a forward end 21 forming an elevated barrel 22 having an axial bore 24 which permits the guidewire 16 to be fed through and straightened. A removable tube 30 defines a bore 23 therethrough for receiving the guidewire 16. Removable tube 30 acts as a straightener element to further straighten the guidewire 16.

The guidewire advancement device 10 further includes an arced handle 26 attached to the underside of longitudinal base 12. The arced handle member 26 cups the middle, ring, and small fingers of the medical professional advancing the guidewire 16. The arced handle 26 includes a forward beveled U-shaped clamp 28 which facilitates the attachment of flexible tube 16 to the arced handle 26.

FIG. 2 is an elevational view of the guidewire advancement device 10 of FIG. 1. FIG. 2 shows the guidewire 16 partially advanced from the guidewire advancement device 10. As shown in FIG. 2, the guidewire 16 is fed from tube 14 through the elevated rear end 13, through the central open section 20 which extends over the longitudinal base 12 and into barrel 22 and through removable tube 30. As shown in FIG. 2, the J-shaped portion 17 of the guidewire 16 returns to its “J” shape after exiting the removable tube 30.

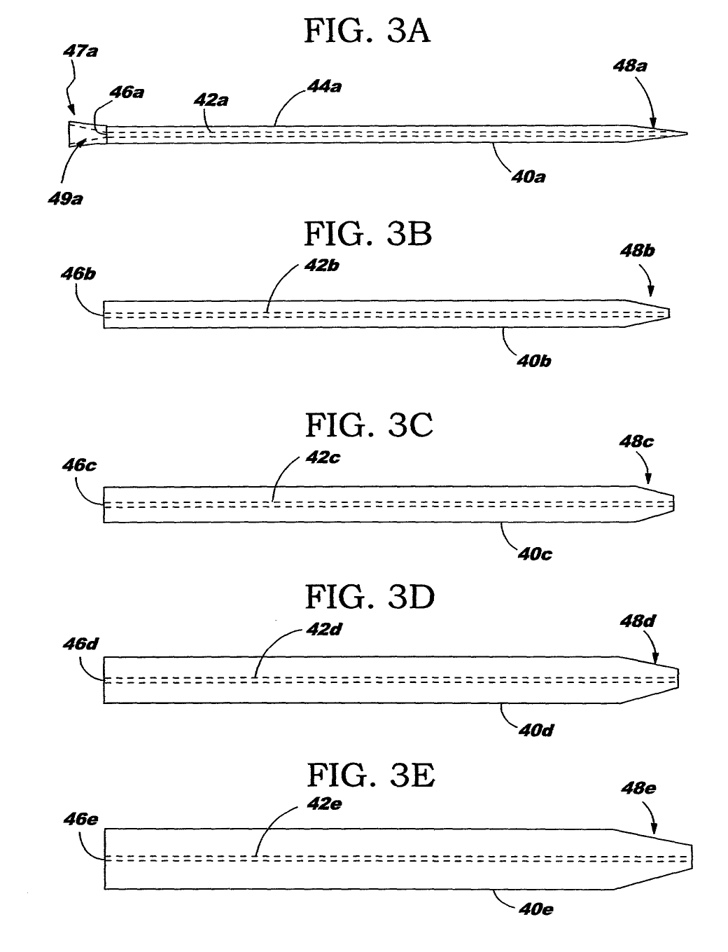

A dilator kit in accordance with the present invention also includes a plurality of dilators. FIGS. 3a-e are elevational views of dilators 40a, 40b, 40c, 40d, 40e in accordance with the present invention. Each dilator is preferably made of a plastic material. Each dilator, e.g., 40a, defines a respective outer surface, e.g. 42a having a respective periphery, e.g. 44a. Each dilator, e.g. 40a, also defines an internal throughbore, e.g. 46a, for receiving the guidewire 16, and a tapered leading end, e.g. 48a. Optionally, each dilator may include a widened flange, e.g. 47a, defining a tapered passageway 49a for guiding the guidewire 16 into the internal throughbore, e.g. 46a. A plurality of dilators included in a kit have peripheries of various dimensions so that a set of dilators may be used as serial dilators; by using the plurality of dilators in sequence from smaller to larger periphery, an orifice is gradually dilated, as discussed further below. For a dilation kit suitable for a PEG tube reinsertion, the largest dilator in the set should have a periphery approximately as large as the periphery of the PEG tube and preferably approximately 1-2 mm larger, e.g. approximately 8 mm in diameter. Each dilator is preferably approximately 10-25 cm in length and most preferably 20 cm in length.

A dilation kit in accordance with the present invention is used as follows to dilate an orifice in biological tissue, e.g. for reinsertion of a PEG tube. First, the guidewire 16 is positioned in the orifice to be dilated, e.g., in a partially closed orifice in a patient's abdomen resulting from dislocation of a PEG tube. Various techniques for positioning a guidewire 16 are well known in the art and any suitable technique may be used. For example, the guidewire 16 is positioned by inserting a first dilator 40a into the orifice, such as an orifice in a patient's abdomen where a PEG tube has been removed. The guidewire 16 is then advanced through the first dilator 40a until the J-shaped portion 17 exits the first dilator 40a and engages biological tissue or until the guidewire 16 is otherwise suitably positioned. For example, straightening element 30 of guidewire advancement device 10 may be inserted into widened flange 47a of first dilator 40a to initiate advancement of the guidewire 16 from the guidewire advancement device 10 and into the first dilator 40a. The first dilator 40a stiffens the guidewire during insertion to facilitate proper placement. The guidewire advancement device 10 and first dilator 40a are then completely removed from the guidewire 16, leaving the guidewire 16 positioned in the orifice.

In one embodiment, the first dilator 40a defines a periphery sufficiently small to prevent substantial dilation of the orifice, e.g. approximately 4 mm in diameter, to facilitate placement of the guidewire without causing injury to the patient due to dilation of the orifice or other biological tissue before the guidewire is properly positioned in the orifice. In this embodiment, the first dilator 40a acts primarily as a straightening element for straightening the “J-shaped” portion and/or for stiffening the guidewire 16 during insertion into the orifice in the biological tissue. This first dilator may be of a type similar to a dilator of a type well known in the art for intravascular procedures. Alternatively, a syringe, a small tube, or similar element could be used as a straightening element and/or an initial dilator.

Next, the orifice is dilated by advancing a second dilator 40b into the orifice. This is performed by inserting a distal end 19 of the guidewire 16 into the throughbore, e.g. 46b, at the leading end 48b of the second dilator 40b. This threads the second dilator 40b onto the guidewire 16. The second dilator 40b is then advanced along the guidewire 16 and into the orifice. The second dilator 40b is selected because it defines an external periphery 42b dimensioned for dilating the orifice. In other words, because the second dilator 40b has an external periphery 42b slightly larger than the orifice, the orifice is caused to be dilated as the dilator is advanced into the orifice. Risk of injury to the patient is minimized by using a small diameter dilator to insert the guidewire into the orifice and by subsequently using the guidewire to precisely position larger dilators in the orifice, e.g., the established PEG tube track through the patient's abdomen.

The second dilator 40b is then retracted from the orifice and along the guidewire 16 until it has been removed from the guidewire 16. The guidewire 16 remains in place in the orifice.

Next, the orifice is further dilated by advancing a third dilator 40c along the guidewire 16 and into the orifice. The third dilator 40c defines an external periphery 42c dimensioned for further dilating the orifice. In other words, the third dilator 40c is slightly larger than the second dilator 40b and/or the orifice after dilation by the second dilator.

The third dilator 40c is then retracted from the orifice and along the guidewire 16, at which point it is removed from the guidewire 16. The guidewire 16 remains in place in the orifice. This process may be repeated for successively larger dilators 40d, 40e, if necessary until the orifice has been sufficiently dilated, e.g., to permit simple percutaneous reinsertion of a PEG tube.

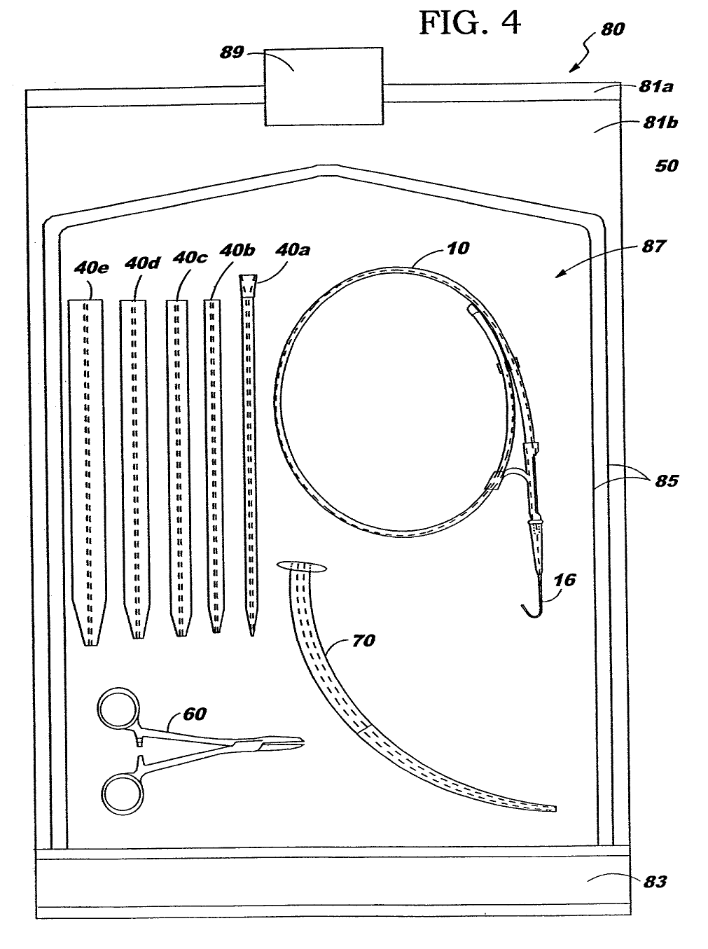

If the objective of the procedure is mere dilation of the orifice, the guidewire 16 may then be removed by simply pulling the guidewire 16 out of the orifice. If the objective of the procedure is to reinsert a PEG tube, a PEG tube is advanced along the guidewire 16 and into the orifice before removing the guidewire 16. An exemplary PEG tube 70 is shown in FIG. 4.

In one embodiment, the dilation kit 50 includes a PEG tube, such as exemplary PEG tube 70. Various configurations of PEG tubes are known in the art. Any suitable PEG tube may be included in the dilation kit 50.

If desired, proper placement of the PEG tube can be confirmed by aspirating gastric contents via the PEG tube, e.g., using a Toomey syringe, or by using radiography to visualize placement of the PEG tube relative to the orifice, e.g. using gastrografin.

In one embodiment, the dilation kit 50 also includes a hemostat. Hemostats of various configurations are well known in the art. FIG. 4 shows a dilation kit 50 in accordance with such an embodiment of the present invention, showing an exemplary hemostat 60. The hemostat 60 may be used to initially dilate the orifice before inserting the guidewire by inserting jaws of the hemostat into the orifice while the hemostat is in a closed position, and subsequently opening the jaws to stretch and/or dilate the orifice. Additionally, the hemostat may be used in a similar manner to dilate the orifice after dilation using the dilation kit as described above.

Preferably, all parts in a dilation kit 50 in accordance with the present invention are packaged together, as in a package of any type well-known in the art. Any suitable package may be used. Because such kits are for medical use, such kits are preferably packaged in a sterile package. The package removably encloses the elements of the kit, i.e., the package is closed to readily retain the elements of the package but should is capable of being easily opened when needed for use. An exemplary package is a “tear away” package 80, as shown in FIG. 4. A suitable example of such a package is made and/or distributed by Medical Action Industries, Inc. under the trade name “SELFSEAL VIEW-PACK” and disclosed in U.S. Pat. No. 4,468,811 to Shaw et al., the disclosure of which is hereby incorporated herein by reference. Such packages are typically made of plastic and/or other common packaging materials. Two layers of packaging material 81a, 81b, are secured at one end 83, e.g. with a high-strength adhesive, and along a perimeter 85, e.g. with a moderate-strength adhesive, to define an internal compartment 87. An overhanging pull-tab 89 is secured to the second layer 81b, such that grasping first layer 81a and pulling the pull-tab 89 breaks the adhesive bond between the first and second layers 81a, 81b along the perimeter 85, and exposes the contents of the package 80 for use.

Having thus described particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A dilation kit for dilating an orifice in living tissue comprising:
a guidewire;
a first dilator defining an internal throughbore for receiving said guidewire and further defining an outer surface having a periphery; and
a second dilator defining an internal throughbore for receiving said guidewire and further defining an outer surface having a periphery greater than said periphery of said first dilator.

2. The dilation kit of claim 1, further comprising a PEG tube.

3. The dilation kit of claim 1, further comprising a hemostat.

4. The dilation kit of claim 1, further comprising a package, said guidewire, said first dilator, and said second dilator being removably enclosed within said package.

5. The dilation kit of claim 4, further comprising a guidewire advancement device, said guidewire advancement device being removably enclosed within said package.

6. The dilation kit of claim 5, wherein said guidewire advancement device comprises:
a longitudinal base having an attached rear end having an opening for feeding a guidewire;
a flexible tube for holding a guidewire and having a proximal end attached to said attached rear end,
said longitudinal base having a forward end affixed to a removable tube defining a bore therethrough for receiving and straightening said guidewire as it is fed from said rear end;
a central open section separating said rear and forward ends for providing finger access to said guidewire so as to advance said guidewire between said rear end and said forward end; and
a handle affixed to the underside of said longitudinal base proximate to said central open section for holding and supporting said guidewire advancement device, said handle configured to cup at least one finger of a user and further having means for securing the distal end of said flexible tube to said handle.

7. The dilation kit of claim 4, wherein said package comprises a tear-away package.

8. The dilation kit of claim 4, further comprising a PEG tube, said PEG tube being removably enclosed within said package.

9. The dilation kit of claim 8, further comprising a hemostat, said hemostat being removably enclosed within said package.

10. The dilation kit of claim 5, said package comprises a tear-away package.

11. A dilation kit for dilating an orifice in living tissue comprising:
a guidewire;
a guidewire advancement device for inserting said guidewire into said orifice;
a first dilator defining an internal throughbore for receiving said guidewire and further defining an outer surface having a periphery; and
a second dilator defining an internal throughbore for receiving said guidewire and further defining an outer surface having a periphery greater than said periphery of said first dilator.

12. The dilation kit of claim 11, wherein said guidewire advancement device comprises a straightening element.

13. The dilation kit of claim 11, wherein said guidewire advancement device further comprises:
a longitudinal base having an attached rear end having an opening for feeding said guidewire;
a flexible tube for holding a guidewire and having a proximal end attached to said attached rear end,
said longitudinal base having a forward end affixed to a removable tube defining a bore therethrough for receiving and straightening said guidewire as it is fed from said rear end;
a central open section separating said rear and forward ends for providing finger access to said guidewire so as to advance said guidewire between said rear end and said forward end; and
a handle affixed to the underside of said longitudinal base proximate to said central open section for holding and supporting said guidewire advancement device, said handle configured to cup at least one finger of the user and further having means for securing the distal end of said flexible tube to said handle.

14. The dilation kit of claim 12, wherein said means for securing is a clamp.

15. The dilation kit of claim 12, wherein said handle includes an arcuate surface extending downwardly and forwardly which forms a curved surface so as to cup at least one finger of the user, thereby providing support for the guidewire advancement device during advancement of the guidewire.

16. The dilation kit of claim 15, wherein said clamp extends generally rearwardly of said curved surface for securing a distal end portion of said flexible tube thereto.

17. The dilation kit of claim 12, wherein said central open section separating said rear and forward ends for providing finger access to said guidewire includes means for facilitating finger access and movement of said guidewire.

18. The dilation kit of claim 17, wherein said means for facilitating finger access and movement of said guidewire comprises an arced surface.

19. The dilation kit of claim 11, further comprising a hemostat.

20. The dilation kit of claim 11, further comprising a PEG tube.

21. The dilation kit of claim 19, further comprising a PEG tube.

22. The dilation kit of claim 11, further comprising a package and a PEG tube, said guidewire, said guidewire advancement device, said first dilator, said second dilator and said PEG tube being removably enclosed within said package.

23. The dilation kit of claim 11, further comprising a package and a hemostat, said guidewire, said guidewire advancement device, said first dilator, said second dilator and said hemostat being removably enclosed within said package

24. The dilation kit of claim 22, further comprising a package, a PEG tube and a hemostat, said guidewire, said guidewire advancement device, said first dilator, said second dilator, said PEG tube and said hemostat being removably enclosed within said package

25. A method for dilating an orifice in biological tissue, the method comprising the steps of:
(a) positioning a guidewire in said orifice;
(b) dilating said orifice by advancing a first dilator along said guidewire and into said orifice, said first dilator defining an external periphery dimensioned for dilating said orifice;
(c) further dilating said orifice by advancing a second dilator along said guidewire and into said orifice, said second dilator defining an external periphery dimensioned for further dilating said orifice; and
(d) removing said guidewire from said orifice.

26. The method of claim 25, wherein step (b) comprises the steps of:
(e) threading said first dilator onto said guidewire; and
(f) removing said first dilator from said guidewire.

27. The method of claim 26, wherein step (c) comprises the steps of:
(g) threading said second dilator onto said guidewire; and
(h) removing said second dilator from said guidewire.

28. The method of claim 25, further comprising the step of:
(i) stretching said orifice with opened jaws of a hemostat.

29. The method of claim 27, further comprising the step of:
(j) advancing a PEG tube along said guidewire and into said orifice.

30. The method of claim 29, further comprising the step of:
(k) aspirating gastric contents via said PEG tube.

31. The method of claim 30, further comprising the step of:
(1) visualizing by radiography placement of said PEG tube relative to said orifice.
